(19) United States
(12) Patent Application Publication
(10) Pub. No.: US 2005/0033229 A1
(43) Pub. Date: Feb. 10, 2005

(54) Degradable arrangement for injecting aqueous solutions and a process for obtaining it

(76) Inventor: Leonardo Scott, Cordoba (AR)

Correspondence Address: THORPE NORTH & WESTERN, LLP., 8180 SOUTH 700 EAST, SUITE 200, P.O. BOX 1219, SANDY, UT, 84070, US

(21) Appl. No.: 10/846,955

(22) Filed: May 14, 2004

(30) Foreign Application Priority Data
May 16, 2003 (AR) P-03-01-01731

(51) Int. Cl. A61M 5/00

(52) U.S. Cl. 604/110

(57) ABSTRACT

Arrangement comprising a barrel, a plunger and a needle made of hardened animal collagen gelatin providing walls of a thickness sufficient for resisting the action of a solvent for at least twice the average time required for filling and administering a water base medicine or solution. Such solvent softens the walls deforming them a fusing them by contact. A seal made of a material which could not be attacked by the solvent and engaged to the lower portion of the plunger acts with expansion on the barrel walls accentuating the deformation produced by the softening caused by the solvent.

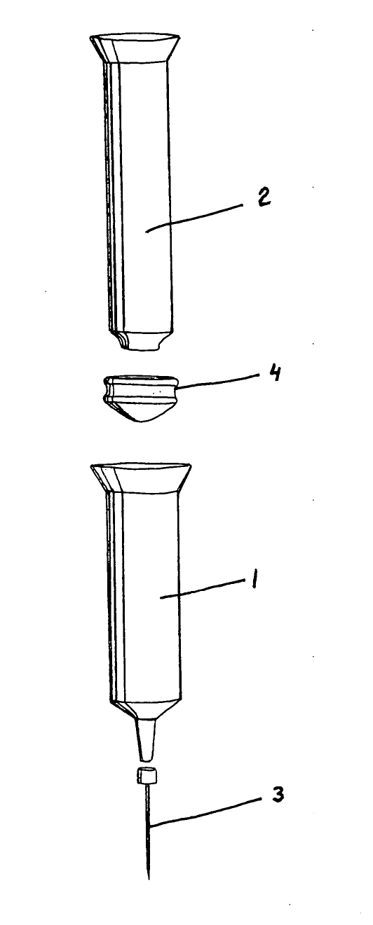

Priority is claimed to Argentina application no. P-03-01-01731, filed May 16, 2003.

### PRIOR ART

U.S. Pat. No. 5,346,929 discloses biodegradable plastics and articles manufactured from such plastics wherein said plastics have a small amount of synthetic plastic polymers, at least a natural (wild) polymer and an agent for attacking said natural polymer leading to degradation of the product.

Such degradation may begin by radiating the article with UV rays, being sufficient the exposition to sunlight, or by adding disintegrating agents to the plastic material constituting the article.

U.S. Pat. No. 4,495,311 discloses incorporating 02 releasing agents which may be protected into hydrophobic polymers to prevent an early release.

An attacking agent may also be introduced, such agent including acid salts such as, for example, ammonium chloride, ammonium sulfate, glycine hydrochloride, alpha-alanine hydrochloride or beta-alanine hydrochloride; such agents may also be encapsulated to prevent early release.

The patent cited in the first place specially claims the use or microorganisms or enzymes as attacking agents.

Further, U.S. Pat. No. 4,976,693 has been considered, which discloses a degradable syringe shaped in a material at least partially soluble in contact with the medicine or solution to be injected.

In this respect, constituted by a barrel, a plunger and a needle, the invention comprises shaping, at least part of the mount, from a material partially soluble into the medicine or the solution, thus resulting in the mount becoming eventually inactivated and the needle used only once.

Thus the mount should comprise a collar for mounting a needle formed, at least partially, from a material which, in contact with the medicine or solution to be injected, is partially degraded to prevent re-utilization of the syringe.

Indirectly, by providing a barrel which will be used afterwards for conveying the sample extracted, or by annulling some of the components, it may be said that re-use of the means protected by the following documents is not possible. Therefore, these documents should be considered part of the art but without affecting the novelty of the instant invention.

WO 01/26550 A1 discloses a device for collecting and metering liquid samples under pressure, particularly for analysis tests and comprises a handle on which a suitable collector is mounted, and a needle at the other end. The handle comprises two valves. The material to be collected flows within the collector provided following the collection needle, its return being not possible.

EP 0824 890 A1 relates to a cylinder within which an aspirator is provided, having a connecting member at its front portion. It provides a support for a double needle the outer end of which is protected by a shield and the inner end engages the connecting member. A region which may be pressed at the lower part modifies the position of the aspirator which advances on the inner needle thus being filled in. When the pressure is increased, this results in exit of the aspirator and disabling of the outer needle.

WO 96/06679 A1 relates to an apparatus having a syringe-shaped body the plunger of which limits a region separating the front portion from the rear portion of the body for limiting mixture between the content of said restricted region with the remaining content.

Finally, FR 2,687,320 discloses a retractile and biodegradable hypodermic syringe enabling recovering and disabling a hypodermic needle after its use.

To this end, once used, the needle retracts automatically being embedded within the barrel thus preventing re-use thereof.

### BRIEF DESCRIPTION OF THE DRAWINGS

The single FIGURE of the drawings is an exploded view showing the different elements of the arrangement of the invention.

### REFERENCES

In the drawing, the same reference numerals indicate the same or corresponding parts. Reference number 1 designates a barrel; number 2 a plunger; number 3 a needle and number 4 a seal.

### DESCRIPTION

The arrangement of the invention is a degradable device for injecting aqueous solutions and is comprised by a barrel, a plunger and a syringe formed from a non synthetic material, degradation of which begins upon contact with water or with aqueous medicines or solutions.

Further, a process for obtaining such arrangement is disclosed.

Operation

The degradable arrangement for injecting aqueous solutions is obtained from a barrel 1, a plunger 2 and a needle 3 made of non-synthetic material, which may be attacked by a solvent, such that upon contact with the solvent the material constituting the arrangement be softened and then deformed.

Upon deformation, none of the mentioned components may be used alone or jointly.

The inventor acknowledges that almost all injection medicines are water base compounds, contrary to very few of them being oil based.

Then inventor has taken into account the amount of the medicine or solution to be injected in order to proceed to the determination of the times elapsed for emptying the barrel 1, concluding that the major portion of injections are administered in amounts up to 5 cc.

It was also considered that water is the universal solvent and available almost all over the world.

Taking into account the above, it is suitable to use a material that, upon contact with water, degrades over a period of time which will be determined by the amount of fluid to be injected, since this in turn determines the thickness of the components to be used.

Therefore, it was concluded that the majority of injected medicines or solutions require a maximum of 60 seconds to fill the barrel and apply the medicine to a patient.

Thus, the inventor determined that a proper wall thickness is that allowing the material to contact the solvent during about twice the time required for injecting the medicine or the solution.

Based on the above, it was concluded that the material suitable for attaining the object of the invention is a hardened animal collagen gelatin.

In fact, by forming the barrel 1, the plunger 2 and the needle 3 from an animal collagen gelatin, the contact with water, by immersion or contact with water contained in water base medicines or solutions to be injected, produces a softening distorting the shape of the different parts.

Such deformation causes that the different parts contact and fuse therebetween.

The mentioned deformation already determines the non-use of the parts, but fusion produced by contact reinforces this feature.

The mentioned deformation begins, as indicated, by contact with water and is accelerated by the expansive pressure exerted by a seal 4 formed from a resilient material.

Seal 4 is fixed to the lower portion of the plunger 2 and allows an effective sweeping of the medicine or solution contained in the barrel 1 in the direction of needle 3, but, being comprised of a resilient material not easily degradable, its action against the inner wall of barrel 1 causes an increase in the expansion deformation of the inner diameter, this diameter not returning to its former size due to loss of molecular memory as a consequence of the mentioned softening.

Applying heat may accelerate deformation initiated as a consequence of the water action.

In fact, the arrangement of the invention was designed in order that, upon contact with the aqueous base of the medicine or solution, it being at the body temperature, it produces an unrecoverable deformation in two minutes from the beginning of said contact.

However, a total destruction of the barrel 1, plunger 2 and needle 3 may be attained by raising the temperature to 45-50° C. In this case, the material of said three elements is dissolved in water.

The components o the instant invention are prepared from a formulation based on animal collagen gelatin, through a series of steps, wherein the first step comprises providing a container with heated animal collagen gelatin.

Such container has the proper size for receiving a sheet to which a plurality of previously refrigerated barrel dies is engaged, such dies being immersed in a second step into the container content during a period of time determined by the thickness of the walls of the barrel 1 to be obtained.

Once the necessary time based on the required wall thickness has expired, the sheet bearing the barrel dies is raised, thus initiating a third cooling and drying step, which will finish with expulsion of the already shaped barrels (1).

Expulsion of barrels is produced preferably insufflating pressurized air into the dies in order that the already shaped barrels (1) fall into a corresponding plurality of receptacles.

At this point, the sheet is in condition of beginning the cycle again thus proceeding to its refrigeration.

In a fourth step, a second sheet provided with a plurality of dies corresponding to the plungers 2, also refrigerated as in the previous step, is immersed into the container with heated collagen for the time necessary to form the wall thickness required by use.

Once the second sheet is removed, in a fifth step cooling and drying of the material adhered to the dies is carried out whereupon, in a sixth step, seals 4 are engaged in the lower end of plungers 2 and, once they are adhered, pressurized air is introduced and plungers are ejected, falling within the barrels 1 retained into the receptacles.

Thus, this second sheet may be cooled to begin the cycle again while during a seventh step the dies provided in a third sheet, suitable for obtaining needles 3, and also previously refrigerated, are introduced into the heated animal collagen gelatin.

As in previous cases, the time for which said dies are immersed is given by the thickness of the material to be obtained and the latter as a function of the time estimated as necessary for effecting the barrel filling and for injecting the medicine or solution.

Once the third sheet is removed, in an eighth step needles 3 are cooled and dried and ejected by introduction of pressurized air into the dies.

When dies are withdrawn, the third sheet is in the proper point for the needles 3 to fall into the plungers 2 already located within barrels 1 arranged into the receptacles, thus completing the arrangement which will now be in condition to be packed and distributed.

In the mentioned process, a sheet provided with barrel dies 1, a second sheet provided with plunger dies and a third sheet provided with needle 3 dies, are mentioned, and all such dies are immersed into heated animal collagen gelatin into a container.

For practical purposes, it is preferred to have a plurality of sheets bearing the different dies, as well as a plurality of containers containing the heated animal collagen gelatin.

Taking into account that the elements obtained through the process described will be all of the same color, it is preferred to have vessels containing heated animal collagen gelatin into which some coloring agent in order to differentiate the different components.

At least, the process disclosed implies having a container with a mixture of heated animal collagen gelatin and a container containing a mixture of heated animal collagen gelatin and a pigment for immersing needle dies 3 in order to differentiate them from the remaining components.

This differentiation is suitable since the three components of the arrangement are stored one inside the other.

Therefore, considering that the three components have the same tone and are translucent, it will be very difficult to distinguish the needle 3 provided the latter does not have some element, for example a different color, allowing to distinguish it when it is packed and stored within the plunger 2.

In a preferred embodiment, in a first stage of said sixth step, a plurality of supports is provided, wherein a corresponding plurality of seals is located (4); in a second stage, said plurality of seals 4 received a suitable adhesive to be fixed to the lower portion of the plunger 2; in a third stage, the second sheet bearing plunger 2 dies is positioned such that the lower ends of said plungers 2 fixed to said dies, contact seals 4 and adhere thereto by means of the mentioned adhesive.

Once the seals 4 are fixed as described, they are removed from the supports and said second sheet bearing plunger dies and now also seals 4 continues with pressurized air introduction.

In order that the plunger bodies may descend within the barrel bodies, and due to the fact that the plungers are provided with said seal 4, said second sheet provided the proper means therefor.

A preferred embodiment consists in that said second sheet descends such as biasing plungers 2 into the respective barrels 1 and only then the pressurized air required for removing the plungers 2 from the dies be introduced.

Once used, the arrangement of the invention is disabled by the deformation produced by the solvent and the expansion action produced by the seal 4 against the walls softened by the water.

However, the action of throwing the arrangement into a container with heated water is preferred, since in this way total dissolution of the barrel 1, the plunger 2 and the needle 3, as well as ejection of the seal 4 are attained.

In this way, a animal collagen gelatin saturated solution is obtained, which may be discarded without biological consequences. The seals 4 may be recycled.

Obviously, such animal collagen gelatin saturated solution could be treated for being re-used; however, this solution would not be economical taking into account the low price thereof.

Expressions such as “medicines” and “aqueous solution” are used to mean any water based injection substance.

The invention has been described in connection with one embodiment thereof and many alternatives and variations will be apparent to those skilled in the art in the light of the foregoing description, its scope being only limited by the annexed claims.

1. Degradable arrangement for injecting aqueous solutions, of the type comprised by a barrel, a plunger and a needle made of a non synthetic material, degradable by contact with a solvent and wherein such plunger has a seal engaged to its lower portion, characterized in that said barrel, plunger and needle are made of a hardened animal collagen gelatin which provides walls of a thickness sufficient for resisting the action of the solvent for a time of at least twice the average time required for filling and administering an aqueous base medicine or solution; said solvent softening said walls deforming them and fusing them by contact therebetween, which seal, acting under expansion on the barrel walls, accentuates deformation thereof.

2. Arrangement as claimed in claim 1, characterized in that the solvent temperature is similar to body temperature.

3. Arrangement as claimed in claim 1, characterized in that it is disposed in water at a temperature of 45-50° C. for producing the total dissolution of the barrel, plunger and the needle, and recovery of the seal.

4. Process for obtaining the degradable arrangement for injecting aqueous solutions according to claim 1, characterized by comprising the steps of:
1) providing a first container filled with heated animal collagen gelatin;
2) immersing a plurality of barrel dies previously refrigerated and engaged to a sheet into the container for a time determined by the thickness of the walls to be obtained;
3) cooling and drying and removal of the shaped barrels housing them into receptacles;
4) immersing the plurality of plunger dies previously refrigerated and engaged to a second sheet, into a heated anima collagen gelatin contained into a second container for a time determined by the thickness of the walls to be obtained;
5) cooling and drying the already shaped plungers;
6) engaging the seals at the lower end of the plungers and removing and housing them into the barrels retained into the receptacles;
7) immersing a plurality of needle dies previously refrigerated and engaged to a third sheet into a heated and pigmented animal collagen gelatin contained into a third container for a time determined by the thickness of the walls to be obtained; and
8) cooling and drying and ejection of the needles into the plungers.

5. Process as claimed in claim 4, characterized in that the sixth step comprises a plurality of steps wherein in a first step a plurality of supports are provided, a corresponding plurality of seals being arranged thereon; in a second step, on said plurality of seals, a proper adhesive is placed and in a third step the lower ends of the plungers engaged with the second sheet dies contact the seals and adhere thereto.

6. Process as claimed in claim 4, characterized in that in the third, sixth and eight steps, the removal of barrels, plungers and needles from the respective dies is produced preferably by introducing pressurized air.

7. Process as claimed in claim 4, characterized in that different pigments are added into each of the containers containing heated animal collagen gelatin.

8. Process as claimed in claim 4, characterized in that in the sixth step the second sheet descends biasing the plungers into the respective barrels before removing them from the respective dies.

9. Process as claimed in claim 4, characterized in that the seal exerts pressure on the barrel wall.
